(19) United States
(12) Patent Application Publication
(10) Pub. No.: US 2011/0306987 A1
(43) Pub. Date: Dec. 15, 2011

(54) Epilation Device

(76) Inventor: Frieder Grieshaber, Neu-Anspach (DE)

(21) Appl. No.: 13/216,907

(22) Filed: Aug. 24, 2011

(30) Foreign Application Priority Data
Mar. 25, 2009 (EP) 09004223.5

(51) Int. Cl. A61B 17/50

(52) U.S. Cl. 606/133

(57) ABSTRACT

The present invention is concerned with an epilation device that comprises a first clamping element having a first clamping surface, a second clamping element having a second clamping surface, the first clamping element and the second clamping element being arranged to be movable between an open position in which a hair or hairs can enter into a gap between the first clamping surface and the second clamping surface and a first closed position, and at least a spacer for defining a distance between the first clamping surface and the second clamping surface in the first closed position. Such an epilation device is specifically suited for plucking out terminal hairs while vellus hair, specifically present on facial skin areas, is plucked out due to its lower thickness that relates to the distance.

### FIELD OF THE INVENTION

The invention concerns an epilation device and in particular an epilation device having a first and a second clamping element where during operation the first clamping element and the second clamping element are movable between an open position in which a hair or hairs can enter into a gap between the first clamping element and the second clamping element and a closed position in which hairs are gripped between the first clamping element and the second clamping element.

### BACKGROUND OF THE INVENTION

Epilation devices according to the opening paragraph are known in the art. Manually operable tweezers are e.g. used to remove facial hairs on a rather individual basis. Power-driven mechanical epilation devices are also known, e.g. WO 2006/037392 A1 describes an epilation device with a motor-driven epilation head that has several tweezers units each having a first clamping element and a second clamping element that are brought in clamping contact during operation of the epilation device.

It is a disadvantage of the known devices that removal of facial hair is performed just in the same way as removal of hairs growing on the legs even though the face is a much more sensitive part of the body.

It is hence desirable to provide an epilation device that is specifically suited for epilation of facial hairs.

### SUMMARY OF THE INVENTION

Such an epilation device that meets above desire is defined by the subject-matter of independent claim 1. Further embodiments are defined by the dependent claims.

The epilation device as proposed comprises a first clamping element having a first clamping surface and a second clamping element having a second clamping surface. The epilation device may comprise a plurality of pairs of first and second clamping elements. The first and the second clamping element are arranged to be movable between an open position in which hairs can enter into a gap between the first clamping surface and the second clamping surface and a first closed position. The epilation device further comprises a spacer for defining a distance between the first clamping surface and the second clamping surface in the first closed position.

The epilation device according to the above description allows to selectively pluck only thick hairs (terminal hairs) and to leave thin hairs (in particular vellus hairs) in the skin. Specifically in face epilation this allows to remove the thick and usually pigmented terminal hairs while to the thin and less pigmented vellus hairs remain in the skin. This leads to less plucking pain during epilation and a natural velvety appearance of the facial skin remains due to the non-plucked vellus hairs (also named “peach fuzz”). The epilation device hence performs selective plucking of hairs.

In an embodiment, the distance defined by the spacer lies in a range of approximately 5 microns and approximately 40 microns. In particular, the distance lies between approximately 10 microns and 30 microns. In certain realizations of this embodiment, the distance that is defined between the clamping surfaces is set to approximately 10 microns or approximately 15 microns or approximately 20 microns or approximately 25 microns or approximately 30 microns. It is to be understood that the distance is a minimum distance between the first and the second clamping surfaces. The distance must not be constant between the first and second clamping surfaces, in particular the first and second clamping surfaces may be separated by a larger distance in some portions. The minimal distance should essentially be achieved on a line extending over the full effective clamping width of the first and second clamping surfaces.

In another embodiment, the spacer is realised by at least an elevation or raised portion present on the first or second clamping element. If more than one such spacer is present, one spacer may be formed on the first clamping element and another spacer may be formed on the second clamping element, The spacers may then be arranged that they together define the distance in the first closed position. In a refinement of this embodiment, the spacer may be realised as an integral portion of the first or second clamping element. In another refinement of this embodiment, the spacer may be formed by a foil or thin sheet affixed (bonded) to the first or second clamping element. This is a relatively simple and cost-efficient realisation of a spacer.

In a further embodiment, the spacer is arranged to be movable between a first spacer position and a second spacer position. In the first spacer position, the first and second clamping elements will achieve the first closed position in which a distance remains between the first and second clamping surfaces. A tight clamping between the first and second clamping surfaces is then inhibited. In the second spacer position, the first and second clamping elements will achieve a second closed position in which the first and second clamping surfaces get into tight clamping contact and no distance remains. By arranging the spacer to be movable, the user can switch the epilation device between a mode in which the epilation device selectively plucks hairs and a mode in which the epilation device performs in a standard manner. Obviously, in the second closed position, a residual distance may remain between the first and second clamping surfaces, which residual distance is the result of, e.g., surface roughness and/or tolerances in the flatness of the first and second clamping elements. Such residual distance will not increase above a few microns, e.g. a distance of 2 microns or 3 microns may remain.

In a refinement of the previous embodiment, the epilation device comprises a spacer actuation unit for moving the spacer between the first spacer position and the second spacer position.

In an even further embodiment, the epilation unit comprises an actuation unit for moving the first and second clamping elements between the open position and the first closed position.

In an embodiment, the epilation device is a powered epilation device, in particular a powered, motor-driven epilation device.

### BRIEF DESCRIPTION OF THE DRAWINGS

The invention will be further elucidated by a detailed description of various embodiments and by reference to figures. In the figures

FIG. 1 is a schematic depiction of an epilation device as proposed;

FIG. 2 is a schematic depiction of a part of an epilation cylinder of an epilation head showing a first embodiment of clamping elements and spacers;

FIG. 3 is a schematic depiction of adjustable spacers;

FIG. 4A is a top view onto a pair of a second embodiment of cooperating clamping elements in a first closed position;

FIG. 4B is front view of the first clamping element according to the second embodiment as shown in FIG. 4A;

FIG. 4C is a top view onto a pair of a third embodiment of cooperating clamping elements in a first closed position; and

FIG. 4D is front view of the first clamping element according to the second embodiment as shown in FIG. 4C.

### DETAILED DESCRIPTION OF THE INVENTION

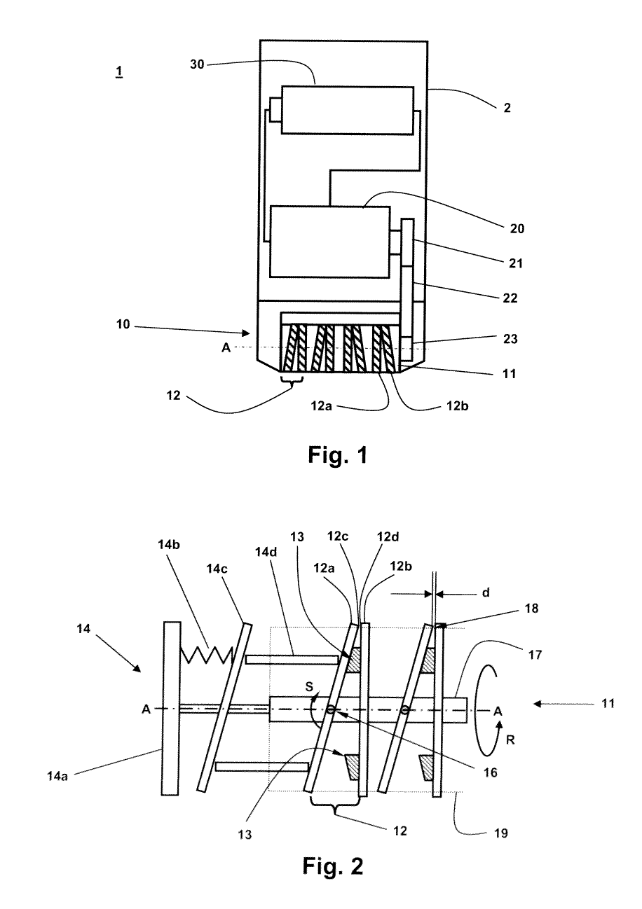

FIG. 1 is a schematic depiction of an epilation device 1 as generally known in the art. The epilation device 1 comprises a housing 2 and a head section 10 that is detachably mounted to the housing 2. Within the housing 2 there is arranged an energy source 30 such as a (rechargeable) battery (e.g. a NiCd battery or a Li-Ion accumulator) and a motor 20 coupled to the energy source 30. In this schematic embodiment, the motor 20 drives a gear wheel 21 that meshes with a transmission gear wheel 22. The head section 10 comprises an epilation cylinder 11 provided to be rotated around its longitudinal axis A that is indicated as a dashed-dotted line. When the head section 10 is attached to the housing 2 the transmission gear wheel 22 meshes with a gear wheel 23 that is coupled to the epilation cylinder 11. During operation a rotary motion is transferred from the motor 20 to the epilation cylinder 11 through the meshed arrangement of gear wheels 21, 22, and 23. The epilation cylinder 11 comprises a plurality of clamping units 12 that each comprise a first clamping element 12a and a second clamping element 12b that are repeatedly moved onto each other to grip hairs and apart to allow hairs to feed into the gap between the first and second clamping elements 12a and 12b as is known in the art. The repeated clamping motion of the first and second clamping elements 12a and 12b is affected while the epilation cylinder 11 is rotating. An epilation cylinder 11 as described is in more detail discussed, e.g., in European Patent No 0 921 744 B1, which is herein incorporated by reference. Another realization of an epilation cylinder where the first and second clamping elements do not extend through the full diameter of the epilation cylinder is e.g. described in International Patent Application No. 2006/037392 A1. which is also herein incorporated by reference. The concrete realization of the epilation cylinder 11 is not relevant for the present application as long as it comprises a first clamping element 12a that is in operation brought into clamping contact with a second clamping element 12b, where one of the first and second clamping elements 12a and 12b could e.g. be realized as a fixedly mounted element or even a integral part of the epilation cylinder 11 itself, e.g. a wall element of an aperture in the epilation cylinder 11.

FIG. 2 is a schematic depiction of a part of an exemplary epilation cylinder 11 of an epilation device as proposed and an actuation unit 14 that is also located in the head section 10 shown in FIG. 1. FIG. 2 is a schematic depiction of an epilation cylinder as described in European Patent No. 0 921 744 B1, which respective content shall be herein incorporated by reference. The outer surface 19 of the epilation cylinder 11 is indicated as a dotted line for sake of clarity. The depicted part of the epilation cylinder 11 has two pairs of first and second clamping elements 12a and 12b that are oppositely arranged to each other. Each of the pairs of first and second clamping elements 12a and 12b are principally arranged and actuated in the same way so that only one such pair is described in more detail. Succeeding pairs of first and second clamping elements 12a and 12b may be angled to each other in a circumferential direction as is described in EP 0 921 744 B1. The first clamping elements 12a extend through the epilation cylinder 11 and are mounted on a central axis 17 so as to be rotatable around a pivot point 16. The pivot point 16 is located on the longitudinal axis A that is again indicated by a dashed-dotted line. The second clamping elements 12b also extend through the epilation cylinder 11 but are fixedly mounted.

The actuation unit 14 comprises abuse plate 14a to which a spring element 14b is mounted that exerts a force on a pressure plate 14c, which pressure plate 14c in turn acts on actuation elements 14d that are mounted in the epilation cylinder 11. When the epilation cylinder 11 rotates during operation in a direction R, the actuation elements 14d, which are provided in cavities in the epilation cylinder 11, slide over the surface of the pressure plate 14c. Due to the spring element 14b, the pressure plate 14c will push the actuation elements 14 into the epilation cylinder as is shown for the upper actuation element 14d in FIG. 2. The actuation element 14d hence acts then on the first clamping element 12a, which will in turn swivel around its pivot point 16 in a swivel direction as indicated by arrow S. In the known devices, as e.g. described in EP 0 921 744 B1, the first clamping element 12a and the second clamping element 12b would then be brought into tight clamping contact where a respective first clamping surface 12c of the first clamping element 12a and a second clamping surface 12d of the second clamping element 12b touch each other without any gap (a gap may result if a hair—or several hairs—is clamped between the first and second clamping elements; without any clamped hairs a gap would not remain). In the epilation device as proposed, a spacer 13 is arranged between the first clamping element 12a and the second clamping element 12b, so that a gap 18 having a (minimal) width d remains between the first clamping surface 12c and the second clamping surface 12d. If the width d is chosen to lie in a range between about 5 micrometer (μm) and 40 μm or in particular in a range between 10 μm and 30 μm, vellus hair that has a typical diameter of below 30 μm will not be efficiently clamped and hence will usually not be plucked from the skin. Only terminal hair that has a larger diameter than vellus hair will be efficiently clamped even though a distance d as defined remains between the first and second clamping surfaces. Typical diameters of a female terminal hair are about 60 μm±20 μm (where hairs have a typical ellipticity of about 1.2-1.7), so that a typical terminal hair is effectively clamped if, e.g., a distance d=30 μm remains between the pair of clamping elements 12a and 12b in the closed position and are thus plucked from the skin. As a result, only the terminal hairs, which often have visible and unaesthetic pigmentation, will be plucked from the skin. A natural (velvety) impression of the skin caused by the remaining vellus hairs is maintained, while terminal hairs considered to have an unaesthetic impression are removed.

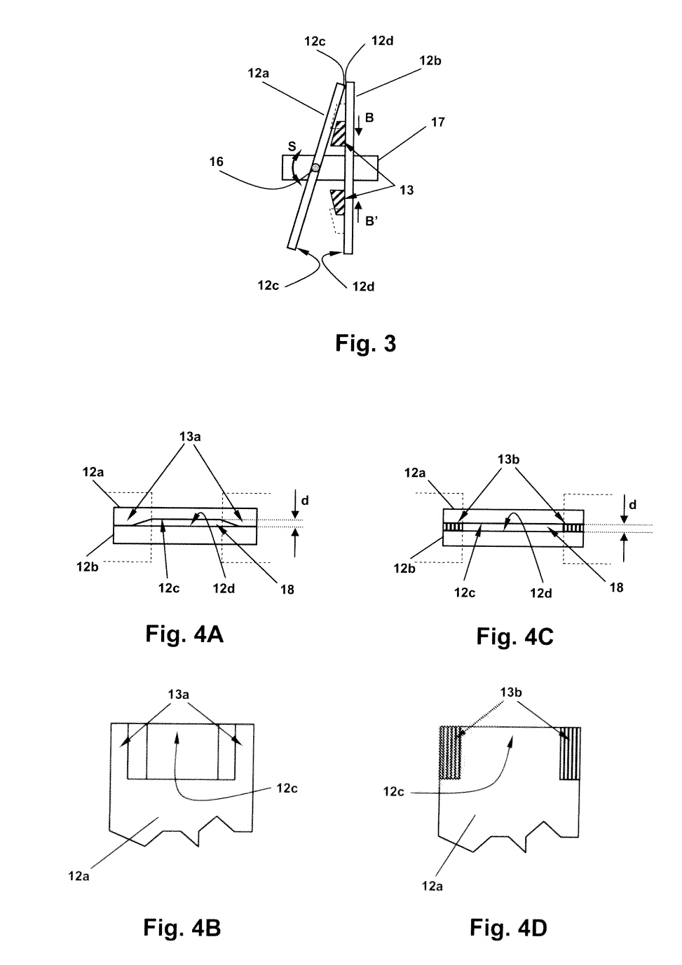

FIG. 3 is a schematic depiction of a pair of a first and a second clamping 12a and 12b as also shown in FIG. 2 but with movable spacers 13. The spacers 13 are each movable in a direction as indicated by arrows B or B′, respectively, from a first spacer position that is indicated by a dashed line (the effect of the spacers 13 in the first spacer position was discussed with reference to FIG. 2, where the spacers are shown in their first spacer position) to a second spacer position. The spacers 13 are shown in FIG. 3 with solid lines in their second spacer position, In the second spacer position, the spacers 13 are moved to a position in which they do not anymore define a distance between the first clamping surface 12c of the first clamping element 12a and the second clamping surface 12d of the second clamping element 12b in the closed position. In this second closed position as shown in FIG. 3, the first clamping element 12a and the second clamping element 12b get in close (or tight, i.e. gap-less) clamping contact with each other. Hence, in an embodiment with movable spacers 13, the user would be able to decide whether the epilation device should be used in a regular manner, in which the first and second clamping element 12a and 12b get in gap-less clamping contact (second closed position) or in a specific facial epilation manner, in which the first and second clamping elements 12a and 12b are spaced from each other in the first closed position due to a spacer 13 defining this distance.

In an alternative embodiment, the spacers could be realized as angularly and radially confined elevations on a disk fixedly mounted on a support cylinder arranged around central axis 17. In an embodiment as shown in FIG. 2. the support cylinder has apertures through which the clamping elements extend. The disks each perpendicularly extend to the support cylinder between a pair of the first and the second clamping elements 12a and 12b. By rotating this support cylinder around the central axis 17 by a certain angle the fixedly mounted disks also rotate and the elevations thereby rotate outside of the clamping elements 12a. 12b so that they do not define a distance d between the first and second clamping elements anymore. Thus, a spacer actuation unit is provided to allow the user to switch between the first spacer position in which the spacers realized as elevations on the disks define a distance between the first and second clamping elements so that the first closed position is achieved and the second spacer position in which the spacers realized as elevations on the disks do not define a distance between the first and second clamping elements anymore so that the second closed position is achieved by rotating the cylinder to which the disks are fixedly mounted.

In another embodiment, the spacers are also provided as movable elements formed on disks that are mounted on the central axis 17 and said disks each extend between a pair of first and second clamping elements. The movable elements are arranged on the disks to be movable between at least a first spacer position in which the movable elements define a distance between the first and second clamping elements an that the first closed position is achieved and a second spacer position in which the spacers realized as elevations do not define a distance between the first and second clamping elements anymore so that the second closed position is achieved. E.g, the spacers realized as movable elements could be guided in undercut grooves in which the first and the second spacer positions are defined by snap-fit recesses into which the movable elements snap when moved in the groove.

FIG. 4A is a schematic top view onto the skin contacting sides of an exemplary embodiment of the first and second clamping elements 12a and 12b and FIG. 4B is a frontal view onto the clamping side of a top area of the first clamping element 12a in the exemplary embodiment as shown in FIG. 4A. FIG. 4A shows the skin contacting sides of the first and second clamping elements 12a and 12b in the first closed position. Spacers 13a are realized as integral parts of the first clamping element 12a by removing material between the spacers 13a in a manufacturing process of the first clamping element 12a. The step of removing material could be done by milling, grinding, etching or any other suitable technique. As a result, the first clamping surface 12c of the first clamping element and the second clamping surface 12d of the second clamping element 12b are spaced by a distance d so that a gap 18 remains in the first closed position. The sides of the clamping elements where the spacers 13a are present could be covered or masked during operation so that vellus hairs are not gripped and plucked by the sides of the first and second clamping elements that still get into gap-less contact in the first closed position. Such a cover is indicated by dashed lines in FIG. 4A.

In the embodiment of an epilation cylinder as discussed, the first and second clamping elements 12a and 12b are brought into the first closed position by pivoting the first clamping element 12a around a pivot point 16 (as shown e.g. in FIG. 2). In the first closed position, the first and second clamping elements 12a and 12b are hence angulated to each other. Hence, the first clamping surface 12c of the first clamping element 12a does not need to be realized by removed material of the first clamping element 12a over the full length of a potentially clamped hair as the angulated arrangement leads to an increasing distance between the first and second clamping elements 12a and 12b in the height direction from the skin contacting side to the central axis 17. As is shown in FIG. 4B, the area of removed material is limited in the height direction, The exact dimensions are depending on the concrete embodiment.

In another embodiment of an epilation cylinder, the first and second clamping elements 12a and 12b may be brought into the first closed position by pushing the first and second clamping elements together without swiveling one of them, i.e. by linearly moving them together. In such an embodiment, the area of removed material would be designed to be as long in the height direction as a typical vellus hair would feed in between the first and second clamping elements.

FIG. 4C is a schematic top view onto the skin contacting sides of another exemplary embodiment of the first and second clamping elements 12a and 12b and FIG. 4D is a frontal view onto the clamping side of a top area of the first clamping element 12a in the exemplary embodiment as shown in FIG. 4C. In this embodiment, spacers 13b are realized by elevated portions, which may in particular be realized by a thin metal or plastic foil glued, welded, or elsewise bonded to the base sheet material of the first clamping element 12a. In the first closed position, which is shown in FIG. 4C, the first clamping surface 12c of the first clamping element 12a and the second clamping surface 12d of the second clamping element 12b are spaced by a distance d so that a gap 18 remains. The sides of the first and second clamping elements 12a and 12b can be covered during operation as is indicated by dashed lines in FIG. 4C so that no hairs are clamped when the sides are brought into gap-less contact.

The embodiments as shown in FIGS. 4A to 4D should not be construed as limiting the invention. In particular, the spacers 13a or 13b realized as integral portions of a clamping element or as foils or thin sheets bonded to a clamping element could be realized on the first and/or the second clamping element and also could be placed at any other position on the clamping side of the first and second clamping elements. In particular, the spacers could be realised in a position more distal to the skin contacting side of the clamping elements such that the skin contacting sides do not partially get in tight clamping contact and a cover can thus be discarded to avoid clamping of vellus hairs at those portions.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is

1. An epilation device comprising:
a first clamping element having a first clamping surface;
a second clamping element having a second clamping surface;
the first clamping element and the second clamping element being arranged to be movable between an open position in which a hair or hairs can enter into a gap between the first clamping surface and the second clamping surface and a first closed position; and
at least a spacer for defining a distance between the first clamping surface and the second clamping surface in the first closed position.

2. The epilation device according to claim 1, wherein the distance lies in a range between approximately 5 microns and approximately 40 microns, in particular between approximately 10 microns and approximately 30 microns.

3. The epilation device according to claim 1, wherein the spacer is formed by at least an elevation or raised portion present on the first clamping element or the second clamping element.

4. The epilation device according to claim 3, wherein the spacer is an integral part of the first clamping element or the second clamping element.

5. The epilation device according to claim 3, wherein the spacer is a foil or thin sheet affixed to the first clamping element or the second clamping element.

6. The epilation device according to claim 1, wherein the spacer is arranged to be movable and has a first spacer position in which the first closed position is obtained during operation and a second spacer position in which a second closed position is obtained during operation in which the first clamping surface and the second clamping surface are in clamping contact with each other essentially without a distance between them.

7. The epilation device according to claim 6 that further comprises a spacer actuation unit for moving the spacer between the first spacer position and the second spacer position.

8. The epilation device according to claim 1 that further comprises an actuation unit for moving the first clamping element and the second clamping element between the open position and the first closed position.

9. The epilation device according to claim I, said epilation device being a powered epilation device.
